(19) United States
(12) Patent Application Publication
(10) Pub. No.: US 2023/0225979 A1
(43) Pub. Date: Jul. 20, 2023

(54) SOLID ORAL COMPOSITIONS COMPRISING COMPOSITE MONOLITHIC MATRICES FOR CHRONOTROPIC ADMINISTRATION OF ACTIVE INGREDIENTS IN THE GASTROINTESTINAL TRACT

(71) Applicant: MOGON PHARMACEUTICALS SAGL, Melide (CH)

(72) Inventor: Massimo Pedrani, Melide (CH)

(73) Assignee: MOGON PHARMACEUTICALS SAGL, Melide (CH)

(21) Appl. No.: 17/998,524

(22) PCT Filed: May 11, 2021
(86) PCT No.: PCT/IB2021/053983

§ 371 Date: Nov. 11, 2022

(30) Foreign Application Priority Data
May 14, 2020 (IT) 1020000011050

(51) Int. Cl. A61K 9/28; A61K 9/20; A61K 9/00; A61K 31/737; A61K 38/40; A61K 31/09; A61K 31/353; A61K 31/05; A61K 31/385; A61K 31/7076; A61K 31/7008; A61K 36/82; A61K 31/122; A61K 31/197; A61K 31/4015; A61K 31/198; A61K 31/145

(52) U.S. Cl. CPC A61K 9/2866; A61K 9/2054; A61K 9/0053; A61K 9/2027; A61K 31/737; A61K 38/40; A61K 31/09; A61K 31/353; A61K 31/05; A61K 31/385; A61K 31/7076; A61K 31/7008; A61K 36/82; A61K 31/122; A61K 31/197; A61K 31/4015; A61K 31/198; A61K 31/145

(57) ABSTRACT

The present invention relates to solid oral compositions with controlled release of active ingredients, comprising a core consisting of a monolithic matrix comprising at least one low-, medium- or high-viscosity hydroxypropyl methylcellulose, or a mixture thereof, a hydroxypropyl cellulose (HPC) and one or more superdisintegrant polymers, and an outer coating of said core consisting of a layer comprising hydroxypropyl methylcellulose and/or ethylcellulose, or of a gastroresistant layer or of a layer comprising ethylcellulose coated in turn with gastroresistant polymers.

The present invention relates to solid oral compositions with controlled release of active ingredients, comprising a core consisting of a monolithic matrix comprising a low-, medium- or high-viscosity hydroxypropyl methylcellulose, or a mixture thereof, hydroxypropyl cellulose (HPC), and one or more superdisintegrant polymers, and an outer coating of said core consisting of a layer comprising hydroxypropyl methylcellulose and/or ethylcellulose, or of a gastroresistant layer or of a layer comprising ethylcellulose coated in turn with gastroresistant polymers.

### PRIOR ART

The use of active ingredients that are pharmacologically active, whether actual medicaments or diet supplements, nutraceuticals or botanical products, requires a pre-established concentration of active ingredients to be reached in vivo, which must be available at the required times and at the specific site of absorption or functionality, also having regard to the body's circadian rhythms. Disorders with a specific circadian cycle exhibit a marked change in symptoms, with peaks and troughs, during the day.

The design of compositions able to release a medicament or nutraceutical with timing suitable to ensure optimum treatment of symptoms involving circadian variations requires full understanding of the absorption, distribution, metabolisation and elimination thereof. Time-specific and site-specific release is achieved by exploiting variations in pH and/or the different transit times of the medicaments/nutraceuticals in the gastrointestinal apparatus.

Gastric voiding times can be highly variable, depending on the type and amount of food eaten, and the fasting pH remains on average between 1.2 and 3.0. Transit times range from a few minutes to a few hours.

In the small intestine, the pH tends to approach neutrality, and the transit time is more constant (about 3±1 hours), whereas in the colon, pH values can range from 5.5 to neutrality (pH 7.0-7.5), and transit times vary considerably from individual to individual, from a few hours to 24-48 hours.

Various controlled-release formulations based on monolithic, multi-particulate or multi-unit matrix or reservoir systems have been described. The technologies used comprise gastroresistant retard systems; slow-release systems (simple matrices); solely pH-dependent release systems; solely pH-independent release systems; pulsatile-release systems (an immediate-release portion combined with a slow, gradual controlled-release portion with a simple matrix); extended-release systems (simple extended-release matrices); and reservoir systems involving the use of containment polymers, acting as semipermeable membranes.

The known formulations, described, for example, in WO200610640, WO2003101421, WO2009125981 and WO2011106416, are mainly characterised by single-component systems wherein the release control effect is determined by a single type of excipient. This can lead to low precision of release of the active ingredient in the site and over time, and high variability of release both in vitro and in vivo.

The common retard forms (gastroresistant and/or lag-time) can also exhibit erratic release in the gastrointestinal tract in the distal part of the ileum and/or the initial part of the colon, rapidly releasing the active ingredient without homogeneous distribution thereof in the gastroenteric, ileocolonic and colonic tracts.

WO200400280, WO2010100657, WO200658059 and WO200658059 report examples of matrices containing both a hydroxypropyl methylcellulose and an acrylic polymer. US20100285125 generically indicates the possibility of obtaining a complex matrix containing different types of hydroxypropyl methylcellulose in a mixture with one or more enteric polymers. However, the formulations actually exemplified are characterised by hydroxypropyl methylcellulose acetate, succinate and phthalate matrices not mixed with acrylic polymers/copolymers and/or shellac.

WO2011069076 discloses sustained—release tablets comprising a core comprising two different hydroxypropyl celluloses, a hydroxypropyl methylcellulose, superdisintegrant polymers, and a gastroresistant coating of methacrylic polymers.

EP 2 468 264 discloses controlled-release tablets comprising a pH-dependent coating and a core consisting of a hydrophilic matrix containing two different hydroxypropyl methylcelluloses.

### DESCRIPTION OF THE INVENTION

It has now been found that the activity of active ingredients, including nutraceuticals, can be efficiently modulated by reducing their frequency of administration and controlling their release in particular sites of the gastrointestinal tract, using composite matrices consisting of a combination of polymers/materials with different characteristics.

In particular, it has been found that the use of a hydroxypropyl methylcellulose with low, medium or high viscosity, and preferably of at least two hydroxypropyl methylcelluloses having different viscosities, in combination with hydroxypropyl cellulose (HPC) and superdisintegrant copolymers (such as croscarmellose sodium, sodium starch glycolate and crosslinked polyvinylpyrrolidone), makes it possible to prepare formulations that overcome the limitations of the previously known formulations.

The solid oral controlled-release nutraceutical compositions according to the invention comprise one or more active ingredients in a core, and an outer coating of said core, wherein:

a) the core consists of:
(i) a monolithic matrix containing one or more active ingredients, a hydroxypropyl methylcellulose having a viscosity ranging between 3 and 5000 mPa·s 2% in H₂O at 20° C. and a hydroxypropyl methylcellulose having a viscosity ranging between 13500 and 280000 mPa·s 2% in H₂O at 20° C.,
   hydroxypropyl cellulose (HPC) and at least one or more superdisintegrant polymers/copolymers, or
   (ii) a monolithic matrix as defined in (i) adjacent to an immediate-release layer comprising the same active ingredient as contained in the monolithic matrix;
b) the coating consists of a layer comprising hydroxypropyl methylcellulose and/or ethylcellulose or of a gastroresistant layer or of a layer comprising hydroxypropyl methylcellulose and/or ethylcellulose coated in turn with gastroresistant polymers.

The core can consist of a composite monolithic matrix (i) or a bi-layer system consisting of a composite monolithic matrix (i) adjacent to an immediate-release layer comprising the same active ingredient(s) as in the monolithic matrix.

In yet another embodiment of the invention, coating b) consists of a layer comprising hydroxypropyl methylcellulose and/or ethylcellulose, or consists of a layer comprising hydroxypropyl methylcellulose and/or ethylcellulose coated with gastroresistant polymers.

The superdisintegrant polymers in the matrix are preferably selected from croscarmellose sodium, sodium starch glycolate and crospovidone. Two different superdisintegrants are preferably used. The gastroresistant coating can be the conventional type, and typically comprises methacrylic acid copolymers soluble at pH≥5.5, pH≥6.0 or pH≥7.0, available on the market (Eudragit, Eudraguard). The preferred combination is polymethacrylate L100 with polymethacrylate S100 at the ratio of 1:10-10:1 (preferably 1:1), soluble at pH≥6.0 and at pH≥7.0; or polymethacrylates L 100/55 soluble at pH≥5.5; or Eudraguard, or shellac; or cellulose acetophthalates/succinates.

In the compositions according to the invention, the hydroxypropyl methylcelluloses constitute 1 to 40% of the weight of the core, hydroxypropyl cellulose constitutes 1 to 30% of the weight of the core, and the polymer/copolymer and/or mixture of superdisintegrants constitutes 0.1 to 20% of the weight of the core. The superdisintegrant polymer is preferably selected from crospovidone, croscarmellose and sodium starch glycolate.

Hydroxypropyl methylcelluloses with low, medium and high viscosity are available on the market under different tradenames (such as Methocel or Hypromellose) K3LV, K100 LV, K250, K750, K1500, K4M, K15M, K35M, K100M, K200M.

The core contains two hydroxypropyl methylcelluloses having different viscosities, more preferably a hydroxypropyl methylcellulose having a viscosity ranging between 3 and 5000 mPa·s 2% in H₂O at 20° C. and a hydroxypropyl methylcellulose having a viscosity ranging between 13500 and 280000 mPa·s 2% in H₂O at 20°, in percentages ranging from 1 to 30% of the weight of the core, preferably 5%.

Ethylcellulose is present in the core-coating layer in percentages ranging from 1% to 20% of the weight of the core; preferably 3-10%.

The matrix core can comprise conventional excipients such as diluents (microcrystalline cellulose, starches, sugars), binders (PVP, starches, cellulose, dextrins, maltodextrins, low-viscosity cellulose), glidants (colloidal silicon dioxides, talc), lubricants (Mg stearate, fumaryl stearate, stearic acid) and other functional excipients (waxes, polycarbophil, carbomer, glycerides).

The matrix is prepared by processes of partition and direct compression, dry granulation, compacting, wet granulation, melting and extrusion.

The resulting matrix/mini-matrix can then be coated with a gastroresistant film containing pH-dependent polymers that prevent release for at least 2 hours under pH conditions <1.2-5.5. The following can be used for this purpose: pH-dependent methacrylic acid copolymers soluble at pH≥5.5 (L 100-55/L 30 D-55); pH-dependent methacrylic acid copolymers soluble at pH 6.0-7.0 (L 100/L 12.5); pH-dependent methacrylic acid copolymers soluble at pH≥7.0 (S 100/S 12.5/FS 30D); shellac; cellulose acetate phthalate; cellulose succinate, methacrylic acid copolymers and starches (Eudraguard Control, Protect, Natural, GRS, Biotic).

At a third stage, a core coating can be applied which is alternative and/or additional to and beneath the gastroresistant coating with pH-independent polymers (ethylcellulose or hydroxypropyl methylcellulose with different viscosities), which act as membranes delaying the passage of the ingredient loaded into the matrix/mini-matrix core following contact with biological fluids (Nutrateric, Surelease, NS Enteric).

The matrix is coated with an amount of polymer sufficient to guarantee that it remains intact in gastric and enteric juices for at least 2-4 hours before the release of the active ingredient from the core (lag time). To reduce the impact of variable gastric voiding times, a further (pH-dependent) gastroresistant coating can be applied outside the (pH-independent) matrix core and outside the (pH-independent) cellulose film coating, to further delay contact between the biological fluids and the modified-release core (extended release).

In this way the system prevents early release during the stomach-jejunum transit time, initiating the modulated-release programme lasting up to 24 hours and ensuring homogeneous distribution of the active ingredient in the duodenum, ileum and distal ileum and in the ascending, transverse and descending tracts of the large intestine.

The use of hydroxypropyl cellulose (HPC) and/or superdisintegrant polymers with different rheological/functional characteristics (viscosity/swelling properties) in combination with hydroxypropyl methylcelluloses allows the release to be modulated for between 4 and 24 hours. If desired, a modified-, controlled-release core can be combined with an immediate-release layer (bi-layer and/or tri-layer matrix/mini-matrix); a system thus designed gives results of “therapeutic equivalence” or different levels of therapeutic efficacy.

Examples of active ingredients usually considered as nutraceuticals which can be advantageously formulated according to the invention comprise chondroitin sulphate, lactoferrin, ubiquinol, quercetin, resveratrol, α-lipoic acid, S-adenosyl methionine (SAMe), glucosamine, green tea, coenzyme Q10, phytosterols, flavonoids, creatine, N-acetylcysteine, glutathione, taurine, lycopene, lutein, zeaxanthin, astaxanthin, vitamin D, vitamin E, vitamin A, vitamin K, gamma oryzanol, isoflavones and melatonin. Medicaments in common use such as anti-inflammatories, analgesics, antibiotics, medicaments active on the central nervous system, antivirals, antidiabetics, hypoglycaemics, immunological, gastroenterological, oncological and cardiovascular medicaments, antihistamines, antidepressants, monoclonal antibodies, bronchodilators, antifungals and antirheumatics can also be used.

The formulations according to the invention are particularly suitable to optimise the absorption, release site and effect of nutraceuticals which have an unfavourable profile in terms of compliance because of the large number of daily administrations and the side effects.

The invention is described in detail in the examples below.

### Example 1

16.65 kg of chondroitin sulphate is loaded into a granulator with 3 Kg of hydroxypropyl cellulose (HPC) and 7.5 Kg of microcrystalline cellulose.

The mixture is granulated with a 5% PVP solution (200 g). The granulate is dried, and 8.3 Kg of hydroxypropyl methylcellulose (HPMC K100 lv), 8.3 Kg of hydroxypropyl methylcellulose (HPMC K4M) and 1.1 Kg of hydroxypropyl methylcellulose (HPMC K100M) are then added in sequence.

The ingredients are mixed until a homogeneous dispersion of active ingredient in the matrices is obtained; 100 g of magnesium stearate, 100 g of talc, 100 g of crosslinked PVP and 100 g of croscarmellose are then added in sequence.

The mixture is then homogenised for at least 15 minutes. This mixture will form part of the first, controlled-release layer of the tablet.

16.65 g of chondroitin sulphate is loaded into a second granulator, and 2.5 Kg of calcium phosphate, 1 Kg of microcrystalline cellulose, 1.16 Kg of crospovidone, 100 g of magnesium stearate and 100 g of talc are added.

The mixture is then homogenised for at least 15 minutes. This mixture will form part of the second, immediate-release layer of the tablet. The two separate mixtures are then compressed to obtain a double-layer tablet weighing 681.2 mg.

The resulting tablets are then film-coated with a solution/suspension based on 1.7 Kg of HPMC 5 premium, 800 g of talc, 230 g of titanium dioxide and 150 g of triethyl citrate, to obtain a tablet with a mean weight of 710 mg.

When subjected to disintegration and dissolution tests at pH≥6.4, the tablets exhibited the following release profile: not more than 60% after 60 minutes, at pH 7.2 not more than 70% after 240 minutes, and not more than 80% after 480 minutes; the value must be >80% after 24 hours.

### Example 2

20 Kg of lactoferrin is loaded into a granulator with 5 Kg of hydroxypropyl cellulose (HPC), 5 Kg of microcrystalline cellulose, 100 g of crosslinked PVP and 100 g of croscarmellose.

The mixture is granulated with a 5% PVP solution (200 g). The granulate is dried, and 8 Kg of hydroxypropyl methylcellulose (HPMC K4M) is then added in sequence.

The ingredients are mixed until a homogeneous dispersion of active ingredient in the matrices is obtained; 150 g of magnesium stearate and 200 g of talc are then added in sequence. The mixture is then compressed to obtain a tablet weighing 386.5 mg. The resulting tablets are then film-coated with a gastroresistant solution/suspension containing 3.2 Kg of shellac (amounting to 800 g of a 25% solution), 650 g of talc, 300 g of titanium dioxide, 150 g of triethyl citrate and 1.45 Kg of HPMC E 5 premium, to obtain a tablet with a mean weight of 420 mg.

When subjected to disintegration and dissolution tests at pH 1, the tablets remain intact for at least 2 hours, with release below 1%; when subjected to the dissolution test at pH≥6.4, they exhibited release below 10%; when subjected to the dissolution test at pH≥7.2, they exhibited the following release profile: not more than 20% after 60 minutes, not more than 60% after 240 minutes, and not more than 80% after 480 minutes; the value must be ≥90% after 24 hours.

### Example 3

20 Kg of ubiquinol are loaded into a granulator with 6.65 Kg of hydroxypropyl cellulose (HPC), 22 Kg of microcrystalline cellulose, 150 g of crosslinked PVP and 150 g of croscarmellose. 10 Kg of hydroxypropyl methylcellulose (HPMC K4M) and 1 Kg of hydroxypropyl methylcellulose (HPMC K100M) are then added in sequence.

The ingredients are mixed until a homogeneous dispersion of active ingredient in the matrix is obtained, and 150 g of magnesium stearate and 250 g of talc are then added in sequence. The mixture is then homogenised for at least 15 minutes.

The mixture is homogenised for at least 20 minutes, followed by compression to obtain a tablet weighing 603.5 mg.

The resulting tablets are then film-coated with a gastroresistant solution/suspension based on 750 g of polymethacrylate (Eudraguard Biotic), 500 g of talc, 200 g of titanium dioxide and 200 g of triethyl citrate, to obtain a tablet with a mean weight of 620 mg.

When subjected to disintegration and dissolution tests at pH 1.2, the tablets remain intact for at least 2 hours, with release below 1%; when subjected to the dissolution test at pH≥6.4, they exhibited release below 1%; when subjected to the dissolution test at pH≥7.2, they exhibited the following release profile: not more than 60% after 60 minutes, not more than 75% after 240 minutes, and not more than 85% after 480 minutes; the value must be >90% after 24 hours.

### Example 4

20 Kg of quercetin is loaded into a granulator with 6.65 Kg of hydroxypropyl cellulose (HPC), 22 Kg of microcrystalline cellulose, 150 g of crosslinked PVP and 150 g of croscarmellose.

4 Kg of hydroxypropyl methylcellulose (HPMC K15M) and 4 Kg of hydroxypropyl methylcellulose (HPMC K100M) are then added in sequence.

The ingredients are mixed until a homogeneous dispersion of active ingredient in the matrix is obtained, and 150 g of magnesium stearate and 250 g of talc are then added in sequence.

The mixture is homogenised for at least 20 minutes, followed by compression to obtain a tablet weighing 573.5 mg.

The resulting tablets are then film-coated with a gastroresistant solution/suspension based on 750 g of polymethacrylate (Eudraguard Biotic), 500 g of talc, 200 g of titanium dioxide and 200 g of triethyl citrate, to obtain a tablet with a mean weight of 590 mg.

When subjected to disintegration and dissolution tests at pH 1.2, the tablets remain intact for at least 2 hours, with release below 1%; when subjected to the dissolution test at pH≥6.4, they exhibited release ≤1%; when subjected to the dissolution test at pH≥7.2, they exhibited the following release profile: not more than 60% after 60 minutes, not more than 75% after 240 minutes, and not more than 85% after 480 minutes; the value must be >90% after 24 hours.

### Example 5

12.5 kg of resveratrol is loaded into a granulator with 3.55 Kg of hydroxypropyl cellulose (HPC) and 12 Kg of microcrystalline cellulose.

10 Kg of hydroxypropyl methylcellulose (HPMC K4M) and 2.5 Kg of hydroxypropyl methylcellulose (HPMC K100M) are then added in sequence.

The ingredients are mixed until a homogeneous dispersion of active ingredient in the matrix is obtained; 150 g of magnesium stearate, 250 g of talc, 125 g of crospovidone (crosslinked??) PVP and 125 g of croscarmellose are then added in sequence.

The mixture is then homogenised for at least 25 minutes. This mixture will form part of the first, controlled-release layer of the tablet, weighing 412 mg.

12.5 Kg of resveratrol is loaded into a second granulator, and 5.25 Kg of calcium phosphate, 750 g of microcrystalline cellulose, 1.25 Kg of crospovidone, 1.25 Kg of croscarmellose, 150 g of magnesium stearate and 250 g of talc are added. The mixture is then homogenised for at least 15 minutes. This mixture will form part of the second, immediate-release layer of the tablet, weighing 214 mg. The two separate mixtures are then compressed to obtain a double-layer tablet weighing 650 mg.

The resulting tablets are then film-coated with a solution/suspension based on 1.5 Kg of polymethacrylate (Eudraguard Control), 500 g of talc, 200 g of titanium dioxide and 200 g of triethyl citrate, to obtain a tablet with a mean weight of 649.5 mg.

The tablets were subjected to disintegration and dissolution tests; when subjected to the dissolution test at pH≥6.4 they exhibited the following release profile: not more than 60% after 60 minutes, at pH 7.2 not more than 70% after 240 minutes, and not more than 80% after 480 minutes; the value must be >80% after 24 hours.

### Example 6

3.75 Kg of α-lipoic acid is loaded into a granulator with 200 g of microcrystalline cellulose and 125 g of hydroxypropyl cellulose (HPC).

250 g of hydroxypropyl methylcellulose (HPMC K4M), 125 g of hydroxypropyl methylcellulose (HPMC K15M), 30 g of crospovidone and 30 g of croscarmellose are then added in sequence.

The ingredients are mixed until a homogeneous dispersion of active ingredient in the matrix is obtained, and the mixture is then granulated with an aqueous solution containing 150 g of polyvinylpyrrolidone (5%).

After drying, 10 g of colloidal silicon dioxide, 30 g of magnesium stearate and 15 g of talc are added in sequence. The mixture is then homogenised for at least 15 minutes.

This mixture will form part of the first, controlled-release layer of the mini-tablet. 3.75 Kg of α-lipoic acid is loaded into a second granulator with 200 g of microcrystalline cellulose, 335 g of crospovidone, 335 g of croscarmellose, 30 g of magnesium stearate and 75 g of talc, and mixed homogeneously.

The mixture is then homogenised for at least 20 minutes. This mixture will form part of the second, immediate-release layer of the mini-tablet.

The two separate mixtures are then compressed to obtain a 5 mm double-layer mini-tablet weighing 94.5 mg.

The resulting mini-tablets are then film-coated with a solution/suspension containing 750 g of HPMC E5 Premium, 200 g of talc and 100 g of triethyl citrate, to obtain a mini-tablet with a mean weight of 105 mg.

When subjected to disintegration and dissolution tests at pH 1, the tablets remain intact for at least 2 hours, with release ≤1%; when subjected to the dissolution test at pH ≥6.4, the tablets exhibited a release not exceeding 50% after 60 minutes; when subjected to the dissolution test at pH≥7.2, they exhibited the following release profile: not more than 70% after 60 minutes; not more than 80% after 240 minutes, not more than 90% after 480 minutes; the value must be >90% after 24 hours.

### Example 7

1.56 Kg of S-adenosyl-methionine (SAMe) is loaded into a granulator with 1.225 Kg of microcrystalline cellulose and 500 g of hydroxypropyl cellulose (HPC).

225 g of hydroxypropyl methylcellulose (HPMC K4M), 225 g of hydroxypropyl methylcellulose (HPMC K15M), 20 g of crospovidone and 20 g of sodium amidoglycolate are then added in sequence. The ingredients are mixed until a homogeneous dispersion of active ingredient in the matrix is obtained. 13 g of magnesium stearate and 22.5 g of talc are then added in sequence. The mixture is then homogenised for at least 15 minutes.

This mixture will form part of the first, controlled-release layer of the mini-tablet.

1.56 Kg of SAMe is loaded into a second granulator.

500 g of microcrystalline cellulose, 225 g of calcium phosphate, 225 g of crospovidone, 225 g of croscarmellose, 13 g of magnesium stearate and 27 g of talc are added and homogeneously mixed.

The mixture is then homogenised for at least 20 minutes. This mixture will form part of the second, immediate-release layer of the mini-tablet.

The two separate mixtures are then compressed to obtain a 4 mm double-layer mini-tablet weighing 65.9 mg.

The resulting mini-tablets are then film-coated with a solution of 14.9 g of HPMC 5 premium, 165.6 g of talc, 29 g of triethyl citrate and 200 g of shellac (25%), to obtain a mini-tablet with a mean weight of 70 mg.

When subjected to the dissolution test at pH 1 and the dissolution test at pH≥6.0, the tablets exhibited the following release profile: not more than 60% after 60 minutes, not more than 75% after 240 minutes, and not more than 85% after 480 minutes; the value must be ≥90% after 24 hours.

### Example 8

3.125 Kg of glucosamine is loaded into a granulator with 1.225 Kg of microcrystalline cellulose and 500 g of hydroxypropyl cellulose (HPC).

225 g of hydroxypropyl methylcellulose (HPMC K4M), 225 g of hydroxypropyl methylcellulose (HPMC K200M), 20 g of crospovidone and 20 g of croscarmellose are then added in sequence. The ingredients are mixed until a homogeneous dispersion of active ingredient in the matrix is obtained. 13 g of magnesium stearate and 22.5 g of talc are then added in sequence. The mixture is then homogenised for at least 15 minutes.

This mixture will form part of the first, controlled-release layer of the mini-tablet.

3.125 Kg of glucosamine is loaded into a second granulator.

500 g of microcrystalline cellulose, 225 g of dicalcium phosphate, 225 g of crospovidone, 225 g of croscarmellose, 13 g of magnesium stearate and 27 g of talc are added and homogeneously mixed. The mixture is then homogenised for at least 20 minutes.

This mixture will form part of the second, immediate-release layer of the mini-tablet.

The two separate mixtures are then compressed to obtain a 4 mm double-layer mini-tablet weighing 97.155 mg.

The resulting mini-tablets are then film-coated with a solution of 15.5 g of HPMC 5 premium, 40 g of talc, 29 g of triethyl citrate, 200 g of polymethacrylate (Eudraguard Biotic) and shellac (25%), to obtain a mini-tablet with a mean weight of 100 mg.

When subjected to the dissolution test at pH 1 and the dissolution test at pH≥6.0, the tablets exhibited the following release profile: not more than 60% after 60 minutes, not more than 75% after 240 minutes, and not more than 85% after 480 minutes; the value must be ≥90% after 24 hours.

### Example 9

15 Kg of green tea is loaded into a granulator with 4.65 Kg of hydroxypropyl cellulose (HPC) and 7.5 Kg of microcrystalline cellulose.

1.1 Kg of hydroxypropyl methylcellulose (HPMC K100lv, 1.1 Kg of hydroxypropyl methylcellulose (HPMC K200M), 10 g of crospovidone and 10 g of croscarmellose are then added in sequence.

The ingredients are mixed until a homogeneous dispersion of active ingredient in the matrix is obtained, and 100 g of magnesium stearate and 100 g of talc are then added in sequence. The mixture is then homogenised for at least 20 minutes. This mixture will form part of the first, controlled-release layer of the tablet.

15 Kg of green tea is loaded into a second granulator, and 2.5 Kg of dicalcium phosphate, 1 Kg of microcrystalline cellulose, 1.16 Kg of crospovidone, 1.16 Kg of croscarmellose, 100 g of magnesium stearate and 100 g of talc are added.

The mixture is then homogenised for at least 15 minutes. This mixture will form part of the second, immediate-release layer of the tablet.

The two separate mixtures are then compressed to obtain a double-layer tablet weighing 522.4 mg.

The resulting tablets are then film-coated with a solution/suspension containing 1.66 Kg of HPMC E5 Premium, 800 g of talc, 200 g of titanium dioxide and 100 g of triethyl citrate, to obtain a tablet with a mean weight of 550 mg.

When subjected to disintegration and dissolution tests at pH 1, the tablets remain intact for at least 2 hours, with release below 1%; at pH≥6.4 they exhibited release ≤10% after 60 minutes; at pH 7.2 release ≤50% after 60 minutes; release ≤60% after 240 minutes, and not more than 80% after 480 minutes; the value must be >90% after 18 hours.

### Example 10

20 Kg of coenzyme Q10 is loaded into a granulator with 4 Kg of hydroxypropyl cellulose (HPC) and 10 Kg of microcrystalline cellulose.

1.1 Kg of hydroxypropyl methylcellulose (HPMC K100lv, 1.1 Kg of hydroxypropyl methylcellulose (HPMC K200M), 10 g of croscarmellose and 10 g of crospovidone are then added in sequence.

The ingredients are mixed until a homogeneous dispersion of active ingredient in the matrix is obtained, and 150 g of magnesium stearate and 200 g of talc are then added in sequence, The mixture is then homogenised for at least 15 minutes. The mixture is then compressed to obtain a tablet weighing 379 mg.

The resulting tablets are then film-coated with a solution/suspension containing 700 g of Nutrateric, 280 g of talc, 300 g of titanium dioxide and 150 g of triethyl citrate, to obtain a tablet with a mean weight of 380 mg.

When subjected to disintegration and dissolution tests at pH 1, the tablets remain intact for at least 2 hours, with release below 1%; at pH≥6.4 they exhibited release below 10%; when subjected to the dissolution test at pH≥7.2, they exhibited the following release profile: not more than 20% after 60 minutes, not more than 60% after 240 minutes, and not more than 80% after 480 minutes; the value must be ≥90% after 18 hours.

### Example 11

20 kg of phytosterols are loaded into a granulator with 2.25 Kg of hydroxypropyl cellulose (HPC) and 7.425 Kg of microcrystalline cellulose.

4.5 Kg of hydroxypropyl methylcellulose (HPMC K100 lv, 4.5 Kg of hydroxypropyl methylcellulose (HPMC K200M), 10 g of crospovidone and 10 g of croscarmellose are then added in sequence.

The ingredients are mixed until a homogeneous dispersion of active ingredient in the matrix is obtained, and 150 g of magnesium stearate and 250 g of talc are then added in sequence.

The mixture is homogenised for at least 20 minutes, followed by compression of the mixture to obtain a tablet weighing 390.95 mg.

The resulting tablets are then film-coated with a solution/suspension containing 700 g of Nutrateric, 305 g of talc, 200 g of titanium dioxide and 200 g of triethyl citrate, to obtain a tablet with a mean weight of 405 mg.

When subjected to disintegration and dissolution tests at pH 1, the tablets remain intact for at least 2 hours, with release below 1%; at pH≥6.4 they exhibited release ≤10% after 60 minutes; at pH 7.2, release ≤60% after 60 minutes; release ≤60% after 240 minutes, and not more than 80% after 480 minutes; the value must be >90% after 18 hours.

### Example 12

20 kg of flavonoids are loaded into a granulator with 2.25 Kg of hydroxypropyl cellulose (HPC) and 7.425 Kg of microcrystalline cellulose. 5.5 Kg of hydroxypropyl methylcellulose (HPMC K100 lv), 3.5 Kg of hydroxypropyl methylcellulose (HPMC K200M), 10 g of crospovidone and 10 g of croscarmellose are added in sequence to the same system.

The ingredients are mixed until a homogeneous dispersion of active ingredient in the matrix is obtained, and 150 g of magnesium stearate and 250 g of talc are then added in sequence.

The mixture is homogenised for at least 20 minutes, followed by compression of the mixture to obtain a tablet weighing 390.95 mg. The resulting tablets are then film-coated with a solution/suspension containing 840 g of Nutrateric, 200 g of talc, 200 g of titanium dioxide and 200 g of triethyl citrate, to obtain a tablet with a mean weight of 405 mg.

When subjected to disintegration and dissolution tests at pH 1, the tablets remain intact for at least 2 hours, with release below 1%; at pH≥6.4 they exhibited release ≤10% after 60 minutes; at pH 7.2 release ≤60% after 60 minutes; release ≤60% after 240 minutes, and not more than 80% after 480 minutes; the value must be >90% after 18 hours.

### Example 13

13.75 kg of creatine is loaded into a granulator with 2.25 Kg of hydroxypropyl cellulose (HPC) and 12 Kg of microcrystalline cellulose.

5 Kg of hydroxypropyl methylcellulose (HPMC K100lv), 5 Kg of hydroxypropyl methylcellulose (HPMC K200M), 10 g of croscarmellose and 10 g of sodium starch glycolate are then added in sequence.

The ingredients are mixed until a homogeneous dispersion of active ingredient in the matrix is obtained, and 150 g of magnesium stearate and 250 g of talc are then added in sequence. The mixture is then homogenised for at least 15 minutes.

This mixture will form part of the first, controlled-release layer of the tablet.

13.75 Kg of creatine is loaded into a second granulator, and 4 Kg of dicalcium phosphate, 750 g of microcrystalline cellulose, 1.25 Kg of crospovidone, 1.25 Kg of croscarmellose, 150 g of magnesium stearate and 250 g of talc are added.

The mixture is then homogenised for at least 20 minutes. This mixture will form part of the second, immediate-release layer of the tablet.

The two separate mixtures are then compressed to obtain a double-layer tablet weighing 598.2 mg.

The resulting tablets are then film-coated with a solution/suspension containing 2.4 Kg of polymethacrylate (Eudraguard Control), 350 g of talc, 200 g of titanium dioxide and 200 g of triethyl citrate, to obtain a tablet with a mean weight of 630 mg.

When subjected to disintegration and dissolution tests at pH 1, the tablets remain intact for at least 2 hours, with release below 1%; at pH≥6.4 they exhibited release ≤5% after 60 minutes; at pH 7.2 release ≤45% after 60 minutes; release ≤60% after 240 minutes, and not more than 85% after 480 minutes; the value must be ≥90% after 18 hours.

### Example 14

7.5 Kg of N-acetyl cysteine is loaded into a granulator with 1.225 Kg of microcrystalline cellulose.

325 g of hydroxypropyl cellulose (HPC), 250 g of hydroxypropyl methylcellulose (HPMC K100 lv), 125 g of hydroxypropyl methylcellulose (HPMC K15M), 30 g of crospovidone and 30 g of croscarmellose are then added in sequence.

The ingredients are mixed until a homogenous dispersion of the active ingredient in the matrix is obtained, and the mixture is then granulated with an aqueous solution containing 150 g of polyvinylpyrrolidone (5%).

After drying, 30 g of magnesium stearate, 10 g of talc and 10 g of colloidal silicon dioxide are added in sequence. The mixture is homogenised for at least 15 minutes, followed by compression of the mixture to obtain a 5 mm mini-tablet weighing 96.9 mg.

The resulting mini-tablets are then film-coated with a solution/suspension containing 630 g of HPMC E5 Premium, 20 g of talc, 100 g of titanium dioxide and 60 g of triethyl citrate, to obtain a mini-tablet with a mean weight of 105 mg.

When subjected to disintegration and dissolution tests at pH 1, the mini-tablets remain intact for at least 2 hours, with release below 1%; at pH≥6.4 they exhibited release ≤10% after 60 minutes; at pH 7.2 release ≤60% after 60 minutes; release ≤70% after 240 minutes, and not more than 85% after 480 minutes; the value must be >90% after 18 hours.

### Example 15

3.125 Kg of glutathione is loaded into a granulator with 1.3 Kg of microcrystalline cellulose.

325 g of hydroxypropyl cellulose (HPC), 225 g of hydroxypropyl methylcellulose (HPMC K100 lv), 225 g of hydroxypropyl methylcellulose (HPMC K15M), 20 g of crospovidone and 20 g of sodium starch glycolate are then added in sequence.

The ingredients are mixed until a homogeneous dispersion of active ingredient in the matrix is obtained.

13 g of magnesium stearate and 47 g of talc are then added in sequence. The mixture is homogenised for at least 15 minutes, followed by compression of the mixture to obtain a 4 mm diameter mini-tablet weighing 91 mg.

The resulting tablets are then film-coated with a solution/suspension containing 710 mg of polymethacrylate (Eudraguard Control), 10 g of talc, 75 g of titanium dioxide and 15 g of triethyl citrate, to obtain a mini-tablet with a mean weight of 100 mg.

When subjected to disintegration and dissolution tests at pH 1, the mini-tablets remain intact for at least 2 hours, with release below 1%; at pH≥6.4 they exhibited release ≤10% after 60 minutes; at pH 7.2 release ≤60% after 60 minutes; release ≤70% after 240 minutes, and not more than 85% after 480 minutes; the value must be >90% after 18 hours.

### Example 16

6.250 Kg of taurine is loaded into a granulator with 1.225 Kg of microcrystalline cellulose.

500 g of hydroxypropyl cellulose (HPC), 225 g of hydroxypropyl methylcellulose (HPMC K100 lv), 225 g of hydroxypropyl methylcellulose (HPMC K15M), 20 g of crospovidone and 20 g of croscarmellose are then added in sequence.

The ingredients are mixed until a homogeneous dispersion of active ingredient in the matrix is obtained.

13 g of magnesium stearate and 47 g of talc are then added in sequence. The mixture is homogenised for at least 15 minutes, followed by compression of the mixture to obtain a 4 mm diameter mini-tablet weighing 91 mg.

The resulting mini-tablets are then film-coated with a solution/suspension containing 834 mg of polymethacrylate (Eudraguard Control), 90 g of talc, 75 g of titanium dioxide and 15 g of triethyl citrate, to obtain a mini-tablet with a mean weight of 90 mg.

When subjected to disintegration and dissolution tests at pH 1, the mini-tablets remain intact for at least 2 hours, with release below 1%; at pH≥6.4 they exhibited release ≤10% after 60 minutes; at pH 7.2 release ≤60% after 60 minutes; release ≤70% after 240 minutes, and not more than 85% after 480 minutes; the value must be ≥90% after 18 hours.

The following tables summarise the qualitative and quantitative compositions of Examples 1-16.

TABLE 1
Tablets of Examples 1-5

| | Chondroitin S. | Lactoferrin | Ubiquinol | Quercetin | Resveratrol |
|---|---|---|---|---|---|
| Active ingredient | (333 mg) | (200 mg) | (200 mg) | (200 mg) | (250 mg) |
| Ingredients of MR core | | | | | |
| API | 166.5 | 200 | 200 | 200 | 125 |
| HPC | 30 | 50 | 66.5 | 66.5 | 35.5 |
| Microcrystalline cellulose | 75 | 50 | 220 | 220 | 120 |
| HPMC k100lv | 83 | | | | |
| HPMC K4 M | 83 | 80 | 100 | | 100 |
| HPMC K15 M | | | | 40 | |
| HPMC K100 M | 11 | | 10 | 40 | 25 |
| Crosslinked PVP | 1 | 0.5 | 1.5 | 1.5 | 1.25 |
| Croscarmellose (AcDisol) | 1 | 0.5 | 1.5 | 1.5 | 1.25 |
| Talc | 1 | 2 | 2.5 | 2.5 | 2.5 |
| Hydrated colloidal silicon dioxide | | | | | |
| PVP | 2 | 2 | | | |
| Mg stearate | 1 | 1.5 | 1.5 | 1.5 | 1.5 |
| Total | 454.5 | 386.5 | 603.5 | 573.5 | 412 |
| Ingredients of IR core | | | | | |
| API | 166.5 | | | | 125 |
| Dicalcium phosphate | 25 | | | | 52.5 |
| Microcrystalline cellulose | 10 | | | | 7.5 |
| Crospovidone | 11.6 | | | | 12.5 |
| Croscarmellose (AcDisol) | 11.6 | | | | 12.5 |
| Talc | 1 | | | | 2.5 |
| Mg stearate | 1 | | | | 1.5 |
| Hydrated colloidal silicon dioxide | | | | | |
| Total | 226.7 | | | | 214 |
| Film-coating ingredients | | | | | |
| Ingredients of MR core | | | | | |
| Talc | 8 | 6.5 | 5 | 5 | 5 |
| Eudraguard Control | | | | | 15 |
| Eudraguard Biotic | | | 7.5 | 7.5 | |
| Shellac | | 8 | | | |
| Titanium dioxide | 2.3 | 3 | 2 | 2 | 2 |
| Triethyl citrate | 1.5 | 1.5 | 2 | 2 | 2 |
| HPMC E5 Prem. | 17 | 14.5 | | | |
| Total | 268.8 | 31.5 | 16.5 | 16.5 | 24 |
| Grand total | 710 | 420 | 620 | 590 | 650 |

TABLE 2
Mini-tablets of Examples 6-8

| | α-lipoic | | |
|---|---|---|---|
| | acid | SAMe | Glucosamine |
| Active ingredient | (600 mg) | (250 mg) | (500 mg) |
| Ingredients of MR core | | | |
| API | 37.5 | 15.625 | 31.25 |
| Microcrystalline cellulose | 2 | 12.25 | 12.25 |
| HPC | 1.25 | 5 | 5 |
| HPMC K4 M | 2.5 | 2.25 | 2.25 |
| HPMC K15 M | 1.25 | 2.25 | |
| HPMC K200 M | | | 2.25 |
| Crospovidone | 0.3 | 0.2 | 0.2 |
| Croscarmellose (AcDisol) | 0.3 | | 0.2 |
| Sodium starch glycolate | | 0.2 | |
| Talc | 0.15 | 0.225 | 0.225 |
| Hydrated colloidal silicon dioxide | 0.1 | | |
| PVP | 1.5 | | |
| Mg stearate | 0.3 | 0.13 | 0.13 |
| Total | 47.15 | 38.13 | 53.755 |
| Ingredients of IR core | | | |
| API | 37.5 | 15.625 | 31.25 |
| Dicalcium phosphate | | 2.25 | 2.25 |
| Microcrystalline cellulose | 2 | 5 | 5 |
| Crosslinked PVP | 3.35 | 2.25 | 2.25 |
| Croscarmellose (AcDisol) | 3.35 | 2.25 | 2.25 |
| Talc | 0.75 | 0.27 | 0.27 |
| Mg stearate | 0.3 | 0.13 | 0.13 |
| Hydrated colloidal silicon dioxide | 0.1 | | |
| Total | 47.35 | 27.775 | 43.4 |
| Film-coating ingredients | | | |
| Talc | 2 | 1.656 | 0.4 |
| Eudraguard Biotic | | | 2 |
| Shellac | | 2 | |
| Titanium dioxide | | | |
| Triethyl citrate | 1 | 0.29 | 0.29 |
| HPMC E5 Prem. | 7.5 | 0.149 | 0.155 |
| Total | 10.5 | 4.095 | 2.845 |
| Grand total | 105 | 70 | 100 |

α-Lipoic acid 8 minitabs 5 mm = 600 mg
SAMe 8 minitabs 4 mm = 250 mg
Glucosamine 8 minitabs 5 mm = 500 m

TABLE 3
Tablets of Examples 9-13

| | Green tea | | | | |
|---|---|---|---|---|---|
| | polyphenols | Coenzyme Q10 | Phytosterols | Flavonoids | Creatine |
| Active ingredient | (300 mg) | (200 mg) | (200 mg) | (200 mg) | (275 mg) |
| Ingredients of MR core | | | | | |
| API | 150 | 200 | 200 | 200 | 137.5 |
| HPC | 46.5 | 40 | 22.5 | 22.15 | 22.5 |
| Microcrystalline cellulose | 75 | 100 | 74.25 | 74.25 | 120 |
| HPMC K 100lv | 11 | 11 | 45 | 55 | 50 |
| HPMC K 200 M | 11 | 11 | 45 | 35 | 50 |
| Crosslinked PVP | 0.1 | | 0.1 | 0.1 | |
| Croscarmellose (AcDisol) | 0.1 | 0.1 | 0.1 | 0.1 | 0.1 |
| Sodium starch glycolate | | 0.1 | | | 0.1 |
| Talc | 1 | 2 | 2.5 | 2.5 | 2.5 |
| Mg stearate | 1 | 1.5 | 1.5 | 1.5 | 1.5 |
| Total | 295.7 | 365.7 | 390.5 | 390.95 | 384.2 |
| Ingredients of IR core | | | | | |
| API | 150 | | | | 137.5 |
| Dicalcium phosphate | 41.5 | | | | 40 |
| Microcrystalline cellulose | 10 | | | | 7.5 |
| Crospovidone | 11.6 | | | | 12.5 |
| Croscarmellose (AcDisol) | 11.6 | | | | 12.5 |
| Talc | 1 | | | | 2.5 |
| Mg stearate | 1 | | | | 1.5 |
| Total | 226.7 | | | | 214 |
| Film-coating ingredients | | | | | |
| Talc | 8 | 2.8 | 3.05 | 2 | 3.5 |
| Eudraguard Control | | | | | 24 |
| Nutrateric | | 7 | 7 | 8.4 | |
| HPMC 5 premium | 16.6 | | | | |
| Titanium dioxide | 2 | 3 | 2 | 2 | 2 |
| Triethyl citrate | 1 | 1.5 | 2 | 2 | 2 |
| Total | 27.6 | 14.3 | 15.05 | 14.4 | 31.5 |
| Grand total | 550 | 380 | 405 | 405 | 630 |

TABLE 4
Mini-tablets of Examples 14-16

| | N- | | |
|---|---|---|---|
| | acetylcysteine | Glutathione | Taurine |
| Active ingredient | (600 mg) | (250 mg) | (500 mg) |
| Ingredients of MR MiniCore | | | |
| API | 75 | 31.25 | 62.5 |
| Microcrystalline cellulose | 12.25 | 12.25 | 12.25 |
| HPC | 3.25 | 5 | 5 |
| HPMC K100lv | 2.5 | 2.25 | 2.25 |
| HPMC K15 M | 1.25 | 2.25 | 2.25 |
| Crospovidone | 0.3 | 0.2 | 0.2 |
| Croscarmellose (AcDisol) | 0.3 | | 0.2 |
| Sodium starch glycolate | | 0.2 | |
| PVP | 1.5 | | |
| Talc | 0.15 | 0.225 | 0.225 |
| Mg stearate | 0.3 | 0.13 | 0.13 |
| Silicon dioxide | 0.1 | | |
| Total | 96.9 | 91 | 91 |
| Ingredients of IR core | | | |
| API | | | |
| Microcrystalline cellulose | | | |
| Dicalcium phosphate | | | |
| Crosslinked PVP | | | |
| Croscarmellose (AcDisol) | | | |
| Talc | | | |
| Mg stearate | | | |
| PVP | | | |
| Total | | | |
| Film-coating ingredients | | | |
| Talc | 0.2 | 0.1 | 0.9 |
| Eudraguard Control | | 7.1 | 8.34 |
| Shellac | | | |
| Titanium dioxide | 1 | 0.75 | 0.75 |
| Triethyl citrate | 0.6 | 0.15 | 0.15 |
| HPMC E5 Prem. | 6.3 | | |
| Total | 8.1 | 9 | 11 |
| Grand total | 105 | 100 | 90 |

N-acetylcysteine 8 minitabs 5 mm = 600 mg
Glutathione 8 minitabs 4 mm = 250 mg
Taurine 8 minitabs 5 mm = 500 mg

### Comparative Example 1—Comparison with Formulations According to WO 2011069076

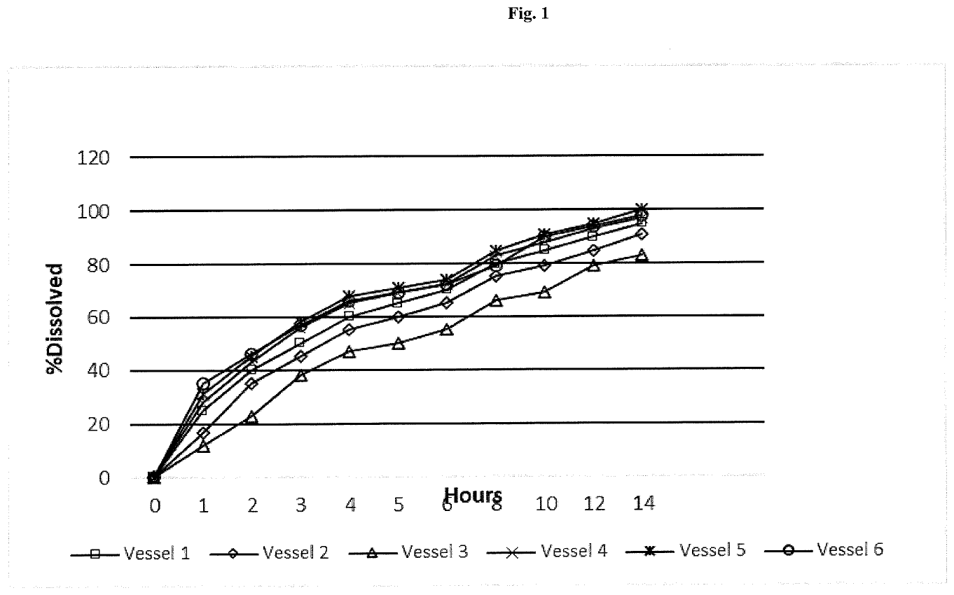

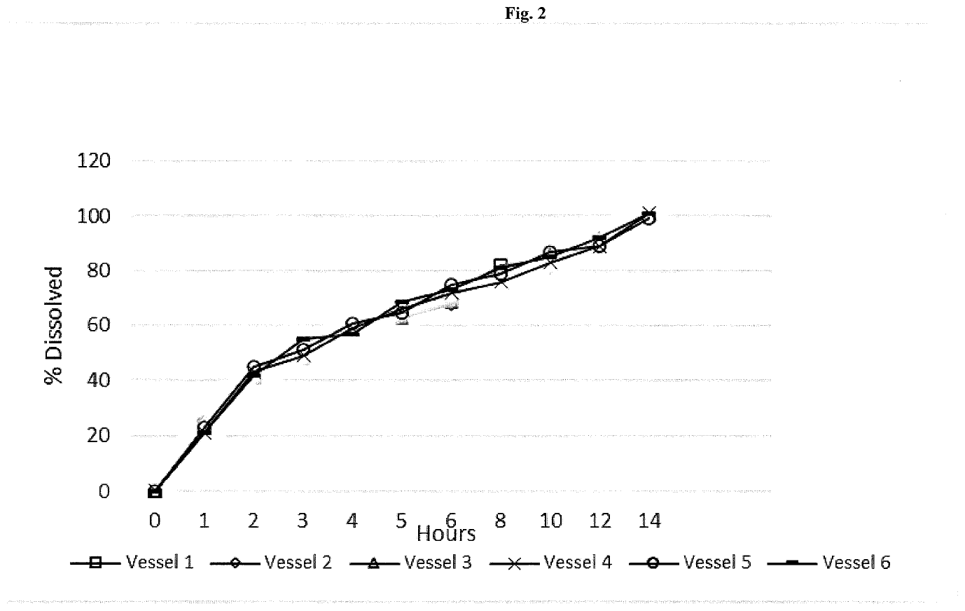

FIGS. 1 and 2 show dissolution profiles representing formulations of donezepil according to Examples 7-16, 19-21, 23, 25 and 27 of WO 2011069076, with formulations according to the invention characterised by the presence of two different superdisintegrants: croscarmellose sodium and crospovidone, each in amounts of 0.5 or 1 mg per tablet. The results deducible from FIGS. 1 and 2 demonstrate that the presence of the two superdisintegrants gives rise to burst effect-free release and greater similarity of behaviour in the dissolution profiles than the formulations of WO 2011069076.

### Comparative Example 2—Comparison with Formulations According to EP 2 468 264

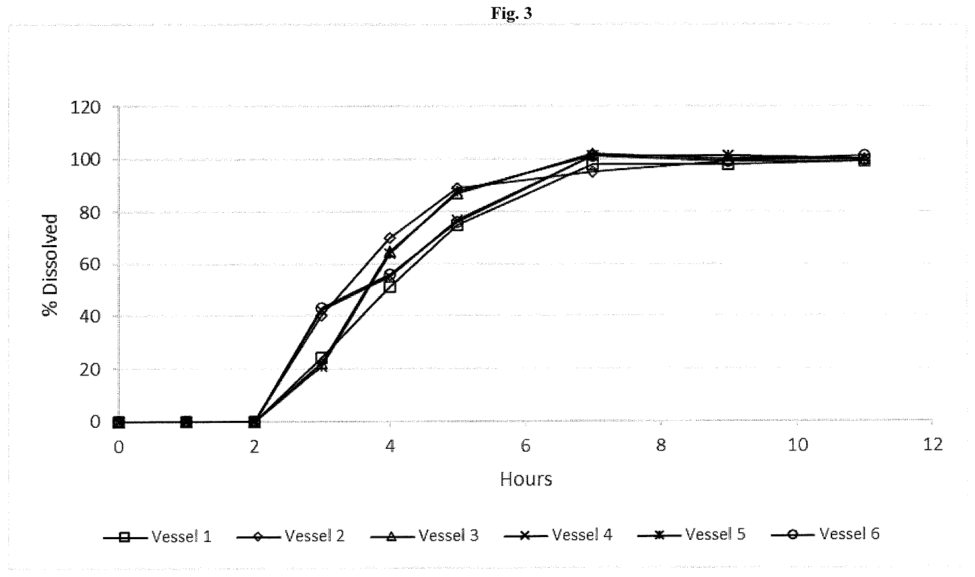

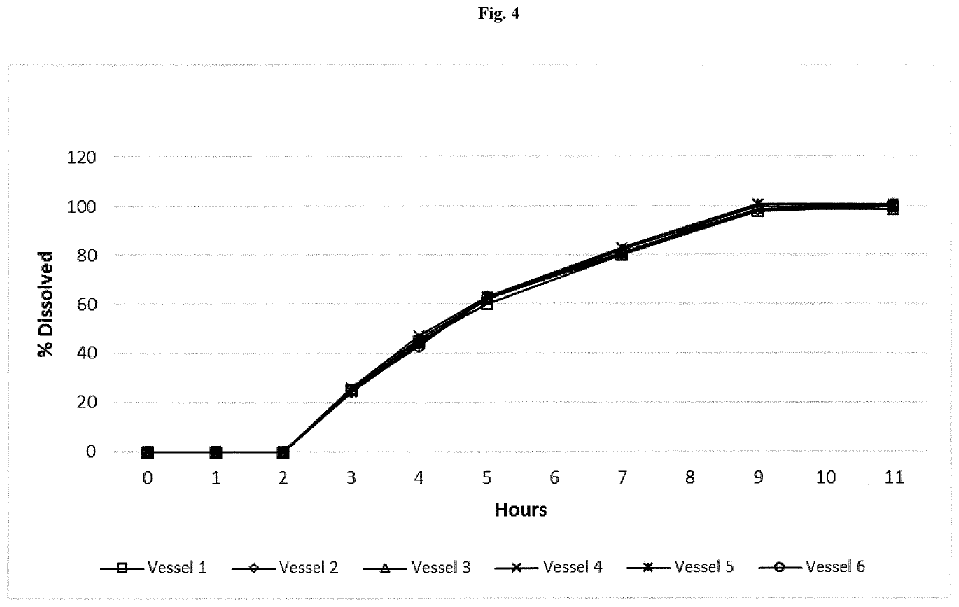

FIGS. 3 and 4 show dissolution profiles representing formulations of mesalazine according to Examples 1-3 of EP 2 468 264, with formulations according to the invention characterised by the presence of two different superdisintegrants: croscarmellose sodium and crospovidone, each in amounts of 6, 8 or 10 mg per tablet. The results deducible from FIGS. 3 and 4 demonstrate that the presence of the two superdisintegrants gives rise to significantly less variability (RSD values) and linearity of behaviour than the formulations of EP 2 468 264.

1. A controlled-release solid oral pharmaceutical composition comprising one or more active ingredients in a core and an outer coating of said core, wherein:
a) the core comprises:
(i) a monolithic matrix containing one or more active ingredients, a hydroxypropyl methylcellulose having a viscosity ranging between 3 and 5000 mPa·s 2% in H₂O at 20° C. and a hydroxypropyl methylcellulose having a viscosity ranging between 13500 and 280000 mPa·s 2% in H₂O at 20° C.
hydroxypropyl cellulose (HPC) and at least two superdisintegrant polymers/copolymers;
or
a monolithic matrix as defined in point (i) adjacent to an immediate-release layer comprising the same active ingredient as contained in the monolithic matrix;
(ii) the outer coating comprises a layer comprising hydroxypropyl methylcellulose and/or ethylcellulose or a gastroresistant layer or a layer comprising hydroxypropyl methylcellulose and/or ethylcellulose coated in turn with gastroresistant polymers.

2. The composition according to claim 1 wherein the core comprises a monolithic matrix as defined in claim 1, point (i).

3. The composition according to claim 1 wherein the core comprises a monolithic matrix as defined in claim 1, wherein said monolithic matrix is adjacent to an immediate-release layer, and wherein said monolithic matrix and said immediate-release layer comprise the active ingredient.

4. The composition according to claim 1, wherein the coating comprises a layer comprising ethylcellulose.

5. The composition according to claim 1, wherein the coating comprises a layer comprising hydroxypropyl methylcellulose and/or ethylcellulose coated with gastroresistant polymers.

6. The composition according to claim 1, wherein the coating comprises a gastroresistant layer.

7. The composition according to claim 1, wherein the acrylic/methacrylic polymer or copolymer is selected from the group consisting of pH-independent methacrylic ester copolymers, pH-independent ammonium alkyl methacrylate copolymers; amino alkyl methacrylate copolymers soluble at pH equal or less than 5.0, methacrylic acid copolymers soluble at pH equal or above 5.5, methacrylic acid copolymers soluble at pH 6.0-7.0 and pH-dependent methacrylic acid copolymers soluble at pH equal or above 7.0.

8. The composition according to claim 7, wherein the gastroresistant layer comprises pH-dependent methacrylic acid copolymers soluble at pH equal or above 5.5; pH-dependent methacrylic acid copolymers soluble at pH 6.0-7.0; pH-dependent methacrylic acid copolymers soluble at pH equal or above 7.0; methacrylic acid polymers and starches, shellac; cellulose acetophthalate; cellulose succinate.

9. The composition according to claim 1, wherein the hydroxypropyl methylcelluloses constitute 1 to 40% of the weight of the core.

10. The composition according to claim 1, wherein the hydroxypropyl cellulose constitutes 0.1 to 30% of the weight of the core.

11. The composition according to claim 1, containing two superdisintegrant polymers selected from crospovidone, croscarmellose and sodium starch glycolate.

12. The composition according to claim 11 wherein the superdisintegrant polymers are present in a percentage ranging from 0.1 to 20% of the weight of the core.

13. The composition according to claim 1, wherein the hydroxypropyl methylcellulose and/or the ethylcellulose are present in percentages ranging from 1 to 20% of the weight of the core.

14. The composition according to claim 1, wherein the active ingredient is selected from chondroitin sulphate, lactoferrin, ubiquinol, quercetin, resveratrol, α-lipoic acid, S-adenosyl methionine (SAMe), glucosamine, green tea, coenzyme Q10, phytosterols, flavonoids, creatine, N-acetyl cysteine, glutathione, taurine, lycopene, lutein, zeaxanthin, astaxanthin, vitamin D, vitamin E, vitamin A, vitamin K, gamma oryzanol, isoflavones and melatonin.
